(19) United States
(12) Patent Application Publication
(10) Pub. No.: US 2024/0024530 A1
(43) Pub. Date: Jan. 25, 2024

(54) ULTRAVIOLET LIGHT EMITTING DEVICE, AND AIR TREATING DEVICE

(71) Applicant: DAIKIN INDUSTRIES, LTD., Osaka (JP)

(72) Inventors: Tomoki SAITO, Osaka (JP); Toshio TANAKA, Osaka (JP); Mamoru OKUMOTO, Osaka (JP)

(73) Assignee: DAIKIN INDUSTRIES, LTD., Osaka (JP)

(21) Appl. No.: 18/478,716

(22) Filed: Sep. 29, 2023

Related U.S. Application Data
(63) Continuation of application No. PCT/JP2022/012729, filed on Mar. 18, 2022.

(30) Foreign Application Priority Data
Mar. 31, 2021 (JP) 2021-060476

(51) Int. Cl. A61L 9/20

(52) U.S. Cl. CPC A61L 9/20; A61L 2209/111; A61L 2209/12; A61L 2209/16

(57) ABSTRACT

An ultraviolet light emitting device includes: an irradiator configured to emit ultraviolet light into a target space (S) where a subject is present; and a recognizer configured to notify the subject of a state of the ultraviolet light emitted from the irradiator.

### TECHNICAL FIELD

The present disclosure relates to an ultraviolet light emitting device and an air treating device.

### BACKGROUND ART

An air conditioner described in Patent Document 1 includes an emitter means configured to emit ultraviolet rays into an installation space, an emission direction changing means configured to change the direction in which the ultraviolet rays are emitted, and a control means. If a moving body is present in the space, the control means controls the emission direction changing means to make the emitter means emit ultraviolet rays into the installation space while avoiding the position of the moving body based on positional information on the moving body. Thus, ultraviolet rays can be emitted into a living space while avoiding a moving body, such as a human body or an animal.

### CITATION LIST

### Patent Document

Patent Document 1: Japanese Unexamined Patent Publication No. 2009-14259

### SUMMARY

A first aspect of the present disclosure is directed to an ultraviolet light emitting device including: an irradiator (20) configured to emit ultraviolet light into a target space (S) where a subject is present; and a recognizer (30) configured to notify the subject of a state of the ultraviolet light emitted from the irradiator (20).

### BRIEF DESCRIPTION OF THE DRAWINGS

FIG. 1 is a diagram schematically illustrating an ultraviolet light emitting device according to a first embodiment.

FIG. 2 is a block diagram of the ultraviolet light emitting device according to the first embodiment.

FIG. 3 is a flowchart showing operations of the ultraviolet light emitting device according to the first embodiment.

FIG. 4 is a diagram schematically illustrating an ultraviolet light emitting device according to a second embodiment, showing a state where a subject is located outside an irradiation region.

FIG. 5 is a diagram schematically illustrating the ultraviolet light emitting device according to the second embodiment, showing a state where a subject is located inside the irradiation region.

FIG. 6 is a perspective view schematically illustrating an ultraviolet light emitting device according to a third embodiment.

FIG. 7 is a diagram schematically illustrating an ultraviolet light emitting device according to a fourth embodiment.

FIG. 8 is a diagram schematically illustrating an ultraviolet light emitting device according to a fifth embodiment.

FIG. 9 is a diagram schematically illustrating an ultraviolet light emitting device according to a fifth embodiment.

### DESCRIPTION OF EMBODIMENTS

Embodiments of the present disclosure will be described in detail below with reference to the drawings. The present disclosure is not limited to the embodiments shown below, and various changes can be made within the scope without departing from the technical concept of the present disclosure. Since each of the drawings is intended to illustrate the present disclosure conceptually, dimensions, ratios, or numbers may be exaggerated or simplified as necessary for ease of understanding.

### First Embodiment

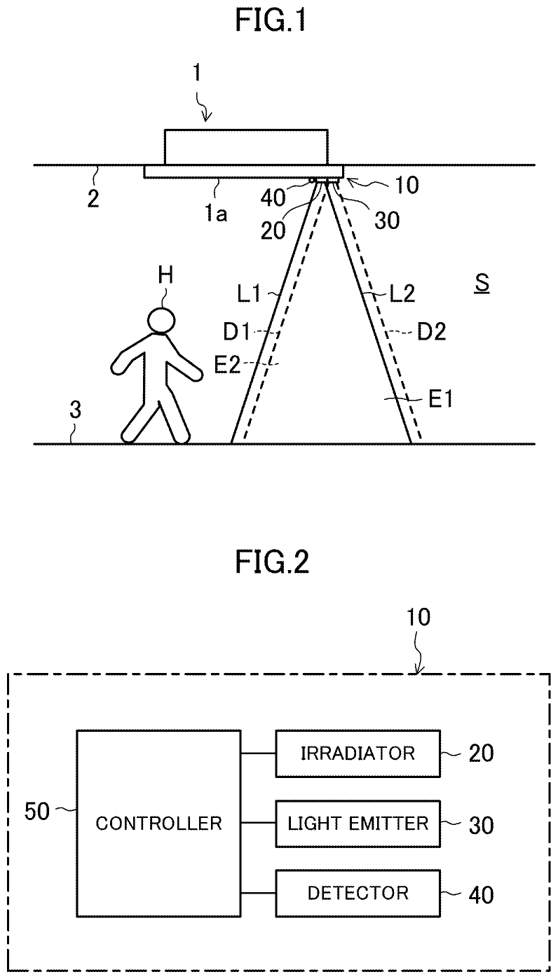

As illustrated in FIG. 1, an ultraviolet light emitting device (10) according to a first embodiment is provided for an air conditioner (1) serving as an air treating device. The air conditioner (1) is of a ceiling-mounted type. Specifically, the air conditioner (1) is of a ceiling-embedded type provided behind a ceiling (2) of a building. The air conditioner (1) may be of a ceiling suspended type. The air conditioner (1) may be of a wall-hanging type or a floor-mounted type. The air conditioner (1) includes a fan and a heat exchanger, which are not shown. The air conditioner (1) adjusts the temperature of air in a target space (S) (e.g., an indoor space).

The ultraviolet light emitting device (10) includes an irradiator (20), a light emitter (30), a detector (40), and a controller (50).

The irradiator (20) emits ultraviolet rays to the target space (S), where a subject is present. The target space (S) includes an indoor space, such as a living space, an office, or a store. The irradiator (20) includes a light source that emits ultraviolet rays. The light source includes a light source element, such as a light-emitting diode (LED) and a laser diode, an excimer lamp, and a mercury lamp. The ultraviolet rays emitted from the irradiator (20) have a wavelength of 190 nm or more and 280 nm or less. Preferably, the ultraviolet rays have a wavelength of 190 nm or more and 230 nm or less.

The irradiator (20) is provided, for example, on a lower surface of a decorative panel (1a) of the air conditioner (1). The irradiator (20) may be provided on a lower surface of the ceiling (2) so as to be adjacent to the air conditioner (1). The irradiator (20) is arranged such that the ultraviolet rays are emitted toward the floor surface (3). The irradiator (20) emits the ultraviolet rays to generate an irradiation region (E1) in the target space (S). In FIG. 1, the irradiation region (E1) is a region between the solid line L1 and the solid line L2.

The light emitter (30) is a recognizer configured to notify a person (a subject (H)) of the state of the ultraviolet rays emitted from the irradiator (20). The light emitter (30) is configured by an LED that emits visible light. The light emitter (30) is provided on the lower surface of the decorative panel (1a) of the air conditioner (1). The light emitter (30) may be provided on the lower surface of the ceiling (2) so as to be adjacent to the air conditioner (1). The light emitter (30) is arranged such that the visible light is emitted toward the floor surface (3). The light emitter (30) emits the visible light to generate a visible light region (E2) in the target space (S). In FIG. 1, the visible light region (E2) is a region between the dashed line D1 and the dashed line D2.

The irradiator (20) and the light emitter (30) are arranged adjacent to each other. The light emitter (30) emits visible light such that the visible light overlaps with the irradiation region (E1) of the ultraviolet rays emitted from the irradiator (20). Preferably, the irradiation region (E1) and the visible light region (E2) entirely coincide with each other. However, only parts of these regions may overlap with each other. The light emitter (30) of the first embodiment notifies the subject (H) of the irradiation region (E1) of the ultraviolet rays as the state of the ultraviolet rays.

The light emitter (30) is configured such that the illuminance and hue of the visible light are changeable. The light emitter (30) notifies the subject (H) of another state of the ultraviolet rays (which will be described in detail later) in accordance with the illuminance and hue of the visible light.

The detector (40) detects the subject (H) in the target space (S). The detector (40) is an infrared or ultrasonic motion sensor. The detector (40) is provided, for example, on the lower surface of the decorative panel (1a) of the air conditioner (1). The detector (40) may be provided on the lower surface of the ceiling (2) so as to be adjacent to the air conditioner (1). The detector (40) detects whether or not the subject (H) is present in the target space (S).

The controller (50) shown in FIG. 2 controls the irradiator (20) and the light emitter (30). The controller (50) includes a microcomputer mounted on a control board, and a memory device (specifically, a semiconductor memory) storing software for operating the microcomputer. The controller (50) may also serve as a controller for the air conditioner (1). The controller (50) may include a server device connected to the irradiator (20) and the light emitter (30) via a network.

The controller (50) controls at least one of the intensity, irradiation amount, or wavelength of the ultraviolet rays emitted from the irradiator (20).

The controller (50) controls the light emitter (30) in accordance with the state of the ultraviolet rays emitted from the irradiator (20). The “state of the ultraviolet rays” as used herein includes the intensity of ultraviolet rays emitted from the irradiator (20), the irradiation amount of the ultraviolet rays, the wavelength of the ultraviolet rays, or the irradiation region (E1) of the ultraviolet rays. The “state of the ultraviolet rays” does not include the ON/OFF state of the irradiator (20).

The irradiation amount of ultraviolet rays [J/cm 2] can be expressed by the following Equation (1).

Irradiation Amount of Ultraviolet Rays [J/cm²]=Intensity of Ultraviolet Rays (Irradiation Intensity) [W/cm²]×Irradiation Time [sec] (1)

The irradiation time [sec] is a time in which the irradiator (20) emits ultraviolet rays.

The controller (50) actuates the light emitter (30) when the detector (40) detects the subject (H). In other words, when the detector (40) detects the subject (H), the light emitter (30) serving as a recognizer notifies the subject (H) of the state of the ultraviolet rays emitted from the irradiator (20).

—Operation of Ultraviolet Light Emitting Device—

An operation of the ultraviolet light emitting device (10) will be described.

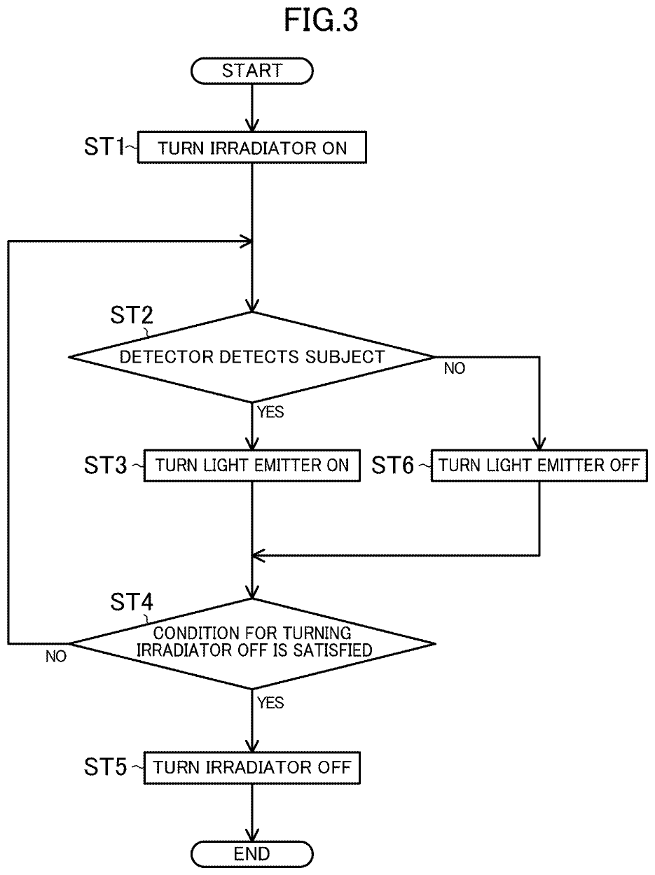

As shown in FIG. 3, the controller (50) turns the irradiator (20) on in step ST1 when the ultraviolet light emitting device (10) starts to operate. The irradiator (20) emits ultraviolet rays from the ceiling (2) toward the floor surface (3).

In step ST2, if the detector (40) detects the subject (H), step ST3 is executed. In step ST3, the controller (50) turns the light emitter (30) on in conjunction with the operation of the irradiator (20). The light emitter (30) emits visible light from the ceiling (2) toward the floor surface (3). The irradiator (20) forms the irradiation region (E1) in a predetermined range of the target space (S).

As illustrated in FIG. 1, the light emitter (30) generates the visible light region (E2) such that the visible light region (E2) overlaps with the irradiation region (E1) generated by the irradiator (20). Thus, the subject (H) can know where in the target space (S) the irradiation region (E1) of ultraviolet rays is present.

The irradiator (20) emits ultraviolet rays with a predetermined intensity when the irradiator (20) is in the on-state. The controller (50) changes the illuminance or hue of the visible light emitted from the light emitter (30) in accordance with the intensity of the ultraviolet rays emitted from the irradiator (20).

Specifically, the light emitter (30) increases the illuminance of the visible light with the increasing intensity of the ultraviolet rays and reduces the illuminance of the visible light with the decreasing intensity of the ultraviolet rays. Alternatively, the light emitter (30) makes the hue of the visible light closer to blue with the increasing intensity of the ultraviolet rays and makes the hue of the visible light closer to red or white with the decreasing intensity of the ultraviolet rays. The subject (H) can know the intensity of the ultraviolet rays emitted from the irradiator (20), based on the illuminance or hue of the visible light from the light emitter (30).

Likewise, the controller (50) may change the illuminance or hue of the visible light emitted from the light emitter (30) in accordance with the wavelength of the ultraviolet rays emitted from the irradiator (20). For example, the light emitter (30) makes the hue of the visible light closer to blue with a decreasing wavelength of the ultraviolet rays and makes the hue of the visible light closer to red or white with an increasing wavelength of the ultraviolet rays. The subject (H) can know the wavelength of the ultraviolet rays emitted from the irradiator (20), based on the illuminance or hue of the visible light from the light emitter (30).

Likewise, the controller (50) may change the illuminance or hue of the visible light emitted from the light emitter (30) in accordance with the irradiation amount of ultraviolet rays emitted from the irradiator (20). For example, the light emitter (30) increases the illuminance of the visible light with an increasing irradiation amount of the ultraviolet rays and reduces the illuminance of the visible light with a decreasing irradiation amount of the ultraviolet rays. Alternatively, the light emitter (30) makes the hue of the visible light closer to blue with an increasing irradiation amount of the ultraviolet rays emitted and makes the hue of the visible light closer to red or white with a decreasing irradiation amount of the ultraviolet rays emitted. The subject (H) can know the irradiation amount of the ultraviolet rays emitted from the irradiator (20), based on the illuminance or hue of the visible light from the light emitter (30).

As can be seen, in step ST3, the light emitter (30), which serves as a recognizer, notifies the subject (H) of the state of ultraviolet light.

If a condition for turning the irradiator (20) off is satisfied in step ST4, the controller (50) turns the irradiator (20) off in step ST5. This condition is, for example, that a predetermined time has elapsed since the irradiator (20) is turned on, or that the subject (H) has turned off the irradiator (20).

If, in step ST3, the detector (40) does not detect the subject (H) with the irradiator (20) turned on, the controller (50) turns the light emitter (30) off in step ST6.

—Features of First Embodiment—

The ultraviolet light emitting device (10) includes the recognizer (light emitter (30)) configured to notify the subject (H) of the state of the ultraviolet light emitted from the irradiator (20) to the target space (S) where the subject is present. Thus, the subject (H) can know the state of the ultraviolet light from the irradiator (20).

The recognizer is the light emitter (30). It is thus possible for the subject (H) to know the state of the ultraviolet light quickly, based on light emitted from the light emitter (30).

The light emitter (30) notifies the subject (H) of the irradiation region (E1) of the ultraviolet light. It is thus possible for the subject (H) to know where the irradiation region (E1) is formed. If the subject (H) is exposed excessively to ultraviolet light, the health of the subject (H) may be impaired. The subject (H) can avoid such a situation by grasping the position of the irradiation region (E1). The active movement of the subject (H) to the irradiation region (E1) enables effective sterilization of the subject (H) through ultraviolet light.

The light emitter (30) notifies the subject (H) of the intensity of the ultraviolet light. Thus, the subject (H) can adjust the time of being exposed to the ultraviolet light, while taking the intensity of the ultraviolet light into consideration.

The light emitter (30) notifies the subject (H) of the wavelength of the ultraviolet light. Thus, the subject (H) can adjust the time of being exposed to the ultraviolet light, while taking the wavelength of the ultraviolet light into consideration.

The light emitter (30) notifies the subject (H) of the irradiation amount of the ultraviolet light. Thus, the subject (H) can adjust the time of being exposed to the ultraviolet light, while taking the irradiation amount of the ultraviolet light into consideration.

The light emitter (30) emits visible light such that the visible light overlaps with the irradiation region (E1) of the ultraviolet light emitted from the irradiator (20) (see FIG. 1). It is thus possible for the subject (H) to grasp the irradiation region (E1) of the ultraviolet light quickly and three-dimensionally.

The light emitter (30) is an LED that emits visible light. The subject (H) can be notified of the state of the ultraviolet light by a relatively simple configuration.

If the detector (40) detects the subject (H), the light emitter (30) notifies the subject (H) of the state of the ultraviolet light. Specifically, if the detector (40) detects the subject (H), the light emitter (30) is turned on. It is thus possible to reduce the chance of the light emitter (30) being turned on when the subject (H) is not present. This can prolong the life of the light emitter (30) and save energy consumption by the light emitter (30).

The ultraviolet light emitted from the irradiator (20) has a wavelength of from 190 nm or more to 280 nm or less. It is thus possible to obtain sufficient sterilization effect through ultraviolet light.

The ultraviolet light emitted from the irradiator (20) has a wavelength of from 190 nm or more to 230 nm or less. Setting the wavelength of the ultraviolet light at or below 230 nm can ease the influence of the ultraviolet light on the human health.

### Second Embodiment

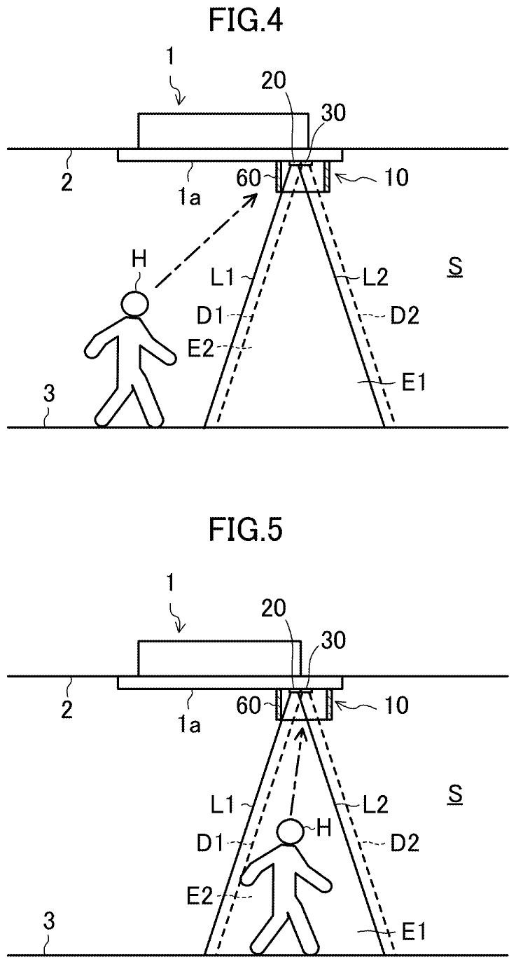

As illustrated in FIGS. 4 and 5, an ultraviolet light emitting device (10) according to a second embodiment includes a shielding member (60). The ultraviolet light emitting device (10) does not have to include a detector (40).

The shielding member (60) shields part of light emitted from the light emitter (30). The shielding member (60) is formed into a tubular shape surrounding the light emitter (30). The shielding member (60) may have a cylindrical shape, a rectangular cylindrical shape, or any other shape.

The shielding member (60) shields the light emitter (30) from the sight of the subject (H) when the subject (H) is outside the irradiation region (E1). The shielding member (60) does no shield the light emitter (30) from the sight of the subject (H) when the subject (H) is inside the irradiation region (E1). In other words, the shielding member (60) disallows the subject (H) outside the irradiation region (E1) to recognize the light emitter (30) visually and allows the subject (H) inside the irradiation region (E1) to recognize the light emitter (30) visually.

The controller (50) turns the light emitter (30) on when the irradiator (20) emits ultraviolet rays. As illustrated in FIG. 4, the shielding member (60) shields the light emitter (30) from the sight of the subject (H) when the subject (H) is outside the irradiation region (E1). The subject (H) can grasp that the subject (H) is outside the irradiation region (E1), based on the fact that the subject (H) cannot visually recognize the light emitter (30).

As illustrated in FIG. 5, the subject (H) can visually recognize the light emitter (30) when the subject (H) is inside the irradiation region (E1). The subject (H) can grasp that the subject (H) is inside the irradiation region (E1), based on the fact that the subject (H) can visually recognize the light emitter (30).

In addition, similarly to the first embodiment, the subject (H) can know the intensity, wavelength, or irradiation amount of the ultraviolet rays, based on the illuminance and hue of the light emitted from the light emitter (30).

Accordingly, it is possible to avoid the excessive exposure of the subject (H) to the ultraviolet rays. The active exposure of the subject (H) to the ultraviolet light enables effective sterilization.

### Third Embodiment

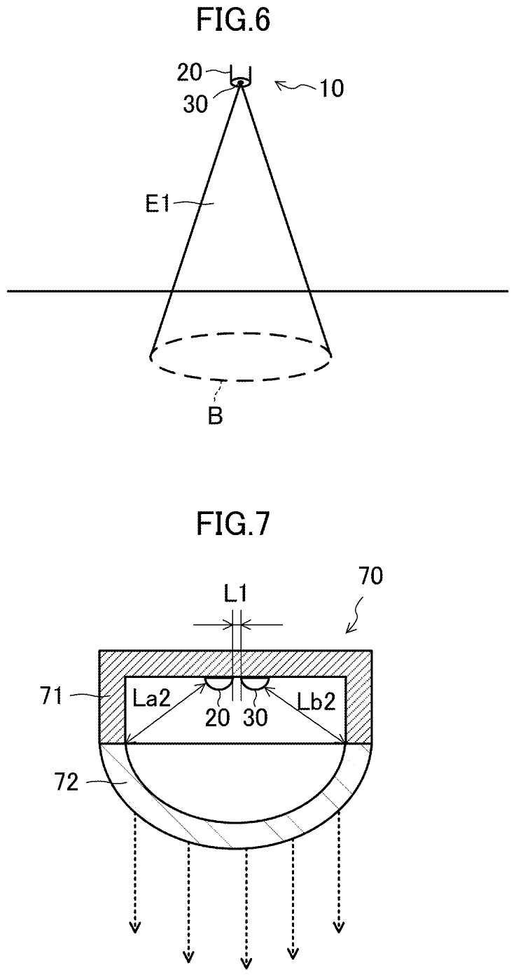

As illustrated in FIG. 6, an ultraviolet light emitting device (10) according to a third embodiment includes a light emitter (30) having a different configuration from the light emitters (30) of the first and second embodiments. The light emitter (30) of the third embodiment emits visible light such that the visible light indicates the boundary of the irradiation region (E1) of ultraviolet light emitted from the irradiator (20). Specifically, the light emitter (30) includes a semiconductor laser element and outputs colored light. The visible light (laser light) emitted from the light emitter (30) is formed along the irradiation region (E1).

Specifically, the light emitter (30) of the third embodiment emits visible light such that a circular boundary line (B) (the dashed line in FIG. 6) is projected onto the floor surface (3). The position of the boundary line (B) corresponds to the boundary of the irradiation region (E1) of the ultraviolet rays. The visible light emitted from the light emitter (30) has a red color, for example.

The subject (H) can know the irradiation region (E1) of the ultraviolet rays by visually recognizing the boundary line (B) on the floor surface (3). In addition, the subject (H) can know the intensity, wavelength, or irradiation amount of the ultraviolet rays, based on the illuminance and hue of light of the boundary line (B).

Accordingly, it is possible to avoid the excessive exposure of the subject (H) to the ultraviolet rays. The active exposure of the subject (H) to the ultraviolet light enables effective sterilization.

### Fourth Embodiment

In a fourth embodiment, an ultraviolet light emitting device (10) includes a lens (72) as a light guide member. As schematically illustrated in FIG. 7, the ultraviolet light emitting device (10) includes a housing (70). The housing (70) accommodates an irradiator (20) and a light emitter (30). The housing (70) includes a base (71) and the lens (72). The base (71) is recessed, and its bottom is provided with the irradiator (20) and the light emitter (30). The irradiator (20) and the light emitter (30) are arranged close to each other.

The lens (72) is attached to the base (71) to cover the opening of the base (71). The refractive index of the lens (72) is set so that the ultraviolet rays emitted from the irradiator (20) and the visible light emitted from the light emitter (30) are guided in the same direction.

Since the ultraviolet rays emitted from the irradiator (20) and the visible light emitted from the light emitter (30) are directed in the same direction (the direction indicated by the dashed arrows in FIG. 7), as described above, the subject (H) can localize the irradiation region (E1) of the ultraviolet rays, based on the visible light.

L1 represents the distance between the irradiator (20) and the light emitter (30); La2 represents the shortest distance between the irradiator (20) and the lens (72); Lb2 represents the shortest distance between the light emitter (30) and the lens (72); and L2 represents the average of the shortest distances La2 and Lb2. In the fourth embodiment, L1 is shorter than L2. Thus, the irradiator (20) and the light emitter (30) are close to each other, making it possible to reduce the chance of the ultraviolet rays and visible light being emitted in different directions. In this regard, it is preferable to cause the irradiator (20) and the light emitter (30) to be in contact with each other.

### Fifth Embodiment

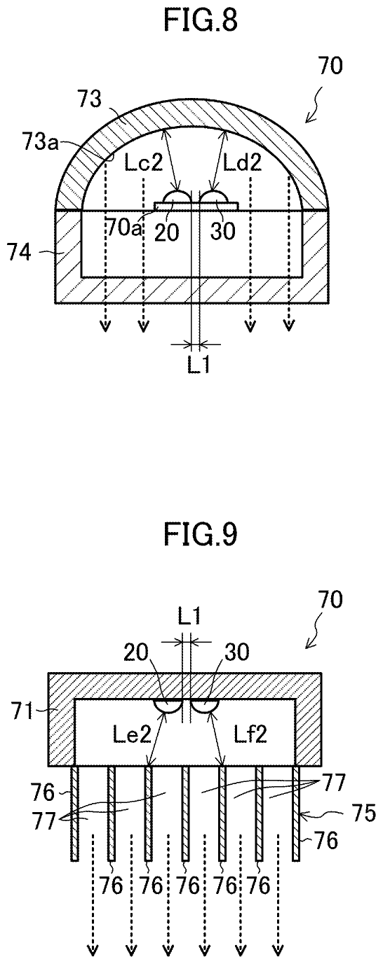

In a fifth embodiment, an ultraviolet light emitting device (10) includes a reflector (73) as a light guide member. As schematically illustrated in FIG. 8, the ultraviolet light emitting device (10) includes a housing (70). The housing (70) accommodates an irradiator (20) and a light emitter (30). The housing (70) includes the reflector (73) and a cover (74). The reflector (73) is formed in the shape of a bowl having one open end. The reflector (73) is made of a metal material that reflects ultraviolet light and visible light. The irradiator (20) and the light emitter (30) are provided inside the reflector (73) via a support plate (70a). The irradiator (20) and the light emitter (30) are arranged close to each other. The irradiator (20) emits ultraviolet rays toward the inner surface (73a) of the reflector (73). The light emitter (30) emits visible light toward the inner surface (73a) of the reflector (73). The reflectance of the reflector (73) is set so that the ultraviolet rays emitted from the irradiator (20) and the visible light emitted from the light emitter (30) are guided in the same direction.

The cover (74) is attached to the reflector (73) to cover the open end of the reflector (73). The cover (74) is made of a translucent material through which light transmits. Instead of the cover (74), the lens (72) described in the fourth embodiment may be employed. When the ultraviolet light emitted from the irradiator (20) and the visible light emitted from the light emitter (30) are reflected off the reflector (73), the ultraviolet light and the visible light are directed in the same direction (the direction indicated by the dashed arrows in FIG. 8) and transmit through the cover (74).

Since the ultraviolet rays emitted from the irradiator (20) and the visible light emitted from the light emitter (30) are directed in the same direction, as described above, the subject (H) can localize the irradiation region (E1) of the ultraviolet rays, based on the visible light.

L1 represents the distance between the irradiator (20) and the light emitter (30); Lc2 represents the shortest distance between the irradiator (20) and the reflector (73); Ld2 represents the shortest distance between the light emitter (30) and the lens (72); and L2 represents the average of the shortest distances Lc2 and Ld2. In the fourth embodiment, L1 is shorter than L2. Thus, the irradiator (20) and the light emitter (30) are close to each other, making it possible to reduce the chance of the ultraviolet rays and visible light being emitted in different directions. In this regard, it is preferable to cause the irradiator (20) and the light emitter (30) to be in contact with each other.

### Sixth Embodiment

In a sixth embodiment, an ultraviolet light emitting device (10) includes a light blocking member (75) as a light guide member. As schematically illustrated in FIG. 9, the ultraviolet light emitting device (10) includes a housing (70). The housing (70) accommodates an irradiator (20) and a light emitter (30). The housing (70) includes a base (71) and the light blocking member (75). The light blocking member (75) includes a plurality of louvers (76). Slits (77) serving as holes through which ultraviolet light and visible light transmit are formed between the louvers (76). The base (71) is recessed, and its bottom is provided with the irradiator (20) and the light emitter (30). The irradiator (20) and the light emitter (30) are arranged close to each other.

The light blocking member (75) is attached to the base (71) to cover the opening of the base (71). The number of the louvers (76), the shape of each louver (76), and the spacing between adjacent louvers (76) of the translucent member (72) are set so that the ultraviolet rays emitted from the irradiator (20) and the visible light emitted from the light emitter (30) are guided in the same direction.

Since the ultraviolet rays emitted from the irradiator (20) and the visible light emitted from the light emitter (30) are directed in the same direction (the direction indicated by the dashed arrows in FIG. 9), as described above, the subject (H) can localize the irradiation region (E1) of the ultraviolet rays, based on the visible light.

L1 represents the distance between the irradiator (20) and the light emitter (30); Le2 represents the shortest distance between the irradiator (20) and the light blocking member (75) (strictly speaking, one of the louvers (76)); Lf2 represents the shortest distance between the light emitter (30) and the lens (72); and L2 represents the average of the shortest distances Le2 and Lf2. In the sixth embodiment, L1 is shorter than L2. Thus, the irradiator (20) and the light emitter (30) are close to each other, making it possible to reduce the chance of the ultraviolet rays and visible light being emitted in different directions. In this regard, it is preferable to cause the irradiator (20) and the light emitter (30) to be in contact with each other.

### Variations

Configurations such as those in the following variations may be employed in the above-described embodiments.

—First Variation—

(1-A) The light emitter (30) may change the illuminance and hue in accordance with the irradiation time of the ultraviolet light emitted from the irradiator (20). The light emitter (30) reduces the illuminance of the visible light as the irradiation time passes. The light emitter (30) may turn off the light when the irradiation time reaches a predetermined time.

(1-B) The light emitter (30) may flash the visible light emitted from the light emitter (30) when the irradiator (20) is switched to the off-state after the elapse of a predetermined time since the irradiator (20) was turned on. It is thus possible for the subject (H) to know the end of the sterilization and disinfection by the irradiator (20) from the flashing visible light.

—Second Variation—

The recognizer may be a display.

(2-A) The display notifies the subject (H) of the state of the ultraviolet rays (such as the intensity, wavelength, irradiation amount, and irradiation region) using letters, symbols, icons, and other elements.

(2-B) In addition, the display may notify the subject (H) that the irradiator (20) is in the on-state. In this case, the display shows letters such as “Disinfecting” to notify the subject (H) of the state. The letters “Disinfecting” may flash on the display. This makes it easier for the subject (H) to know that the irradiator (20) is in the on-state.

(2-C) The display may indicate the intensity, wavelength, or irradiation amount of the ultraviolet rays numerically. The display may indicate the irradiation time of the ultraviolet rays numerically. The irradiation time of the ultraviolet rays may be a continuous irradiation time or a summation of irradiation times during a certain period (e.g., one day). The display may indicate letters “n minutes left until completion of disinfection” (n=1, 2, . . . ).

—Third Variation—

The recognizer may be a sound producer that emits sound.

(3-A) The sound producer may emit sound from a speaker or any other similar component to notify the subject (H) of the state of the ultraviolet rays (such as the intensity, wavelength, irradiation amount, and irradiation region).

(3-B) The sound producer may notify the subject (H), by voice, that the subject (H) is in the irradiation region (E1) when the subject (H) enters the irradiation region (E1) of the ultraviolet rays. The above-described detector (40) detects whether the subject (H) is in the irradiation region (E1).

(3-C) The sound producer may change the volume of sound (an alarm) in accordance with the distance between the subject (H) and the irradiation region (E1). For example, as the subject (H) approaches the irradiation region (E1), the sound producer increases the volume of the sound (alarm). The above-described detector (40) detects the distance between the subject (H) and the irradiation region (E1).

(3-D) The sound producer may emit an alarm in accordance with the time in which the subject (H) is present in the irradiation region (E1). Specifically, if the time in which the subject (H) is present in the irradiation region (E1) exceeds a predetermined time, the sound producer emits an alarm. It is thus possible for the subject (H) to know that the subject (H) is exposed to the ultraviolet rays for a long time.

(3-E) The sound producer may notify the subject (H), by voice, of the irradiation time of the ultraviolet rays. The irradiation time of the ultraviolet rays may be a continuous irradiation time or a summation of irradiation times during a certain period (e.g., one day). The sound producer may make a sound “n minutes left until completion of disinfection” (n=1, 2, . . . ).

(3-F) The sound producer may be provided in a portable terminal owned by the subject (H). The portable terminal includes a cell phone, a smartphone, and a tablet terminal. Entry of the subject into the irradiation region (E1) may cause the portable terminal to make a sound. In this case, for example, wireless communications equipment, such as Bluetooth (registered trademark), may be used to detect the entry of the subject (H) in the irradiation region (E1).

(3-G) The sound producer may emit sound (an alarm) that can be heard by the subject (H) when the subject (H) enters the irradiation region (E1). In this case, the sound producer is disposed near the irradiation region (E1). The sound producer emits an alarm at a relatively low volume.

—Fourth Variation—

The recognizer may be an aroma generator configured to notify the subject (H), by an aroma, of the state of the ultraviolet rays. The aroma generator releases an aroma to notify the subject (H) of the state of the ultraviolet rays.

(4-A) The aroma generator may notify the subject (H) of the state of the ultraviolet rays (such as the intensity, wavelength, irradiation amount, and irradiation region) in accordance with the intensity of the aroma generated.

(4-B) The aroma generator may notify the subject (H), by an aroma, that the subject (H) is in the irradiation region when the subject (H) enters the irradiation region (E1) of the ultraviolet rays. The above-described detector (40) detects whether the subject (H) is in the irradiation region (E1).

(4-C) The aroma generator may change the intensity of an aroma in accordance with the distance between the subject (H) and the irradiation region (E1). For example, as the subject (H) approaches the irradiation region (E1), the aroma generator increases the intensity of the aroma. The above-described detector (40) detects the distance between the subject (H) and the irradiation region (E1).

(4-D) The aroma generator may release an aroma in accordance with the time in which the subject (H) is present in the irradiation region (E1). Specifically, if the time in which the subject (H) is present in the irradiation region (E1) exceeds a predetermined time, the aroma generator releases an aroma. It is thus possible for the subject (H) to know that the subject (H) is exposed to the ultraviolet rays for a long time.

(4-E) The aroma generator may release an aroma when the irradiator (20) is switched to the off-state after the elapse of a predetermined time since the irradiator (20) was turned on. It is thus possible for the subject (H) to know the end of the sterilization and disinfection by the irradiator (20) from the aroma.

—Fifth Variation—

The recognizer may be an air blower configured to blow wind to the subject (H) to notify the subject (H) of the state of the ultraviolet rays.

(5-A) The air blower may notify the subject (H) of the state of the ultraviolet rays (such as the intensity, wavelength, irradiation amount, and irradiation region) in accordance with the strength of the wind.

(5-B) The air blower may blow wind to the subject (H) when the subject (H) enters the irradiation region (E1) of the ultraviolet rays. This enables the subject (H) to know that the subject (H) is in the irradiation region (E1). The above-described detector (40) detects whether the subject (H) is in the irradiation region (E1).

(5-C) The air blower may change the strength of the wind in accordance with the distance between the subject (H) and the irradiation region (E1). For example, as the subject (H) approaches the irradiation region (E1), the air blower increases the strength of the wind. The above-described detector (40) detects the distance between the subject (H) and the irradiation region (E1).

(5-D) The air blower may blow wind to the subject (H) in accordance with the time in which the subject (H) is present in the irradiation region (E1). Specifically, if the time in which the subject (H) is present in the irradiation region (E1) exceeds a predetermined time, the air blower sends wind to the subject (H). It is thus possible for the subject (H) to know that the subject (H) is exposed to the ultraviolet rays for a long time.

(5-E) A fan of the air conditioner (1) may also serve as the air blower.

—Sixth Variation—

The recognizer may be a temperature regulator configured to change the temperature of the air in the target space (S).

(6-A) The temperature regulator may change the temperature of the air of the subject (H) to notify the subject (H) of the state of the ultraviolet rays (such as the intensity, wavelength, irradiation amount, and irradiation region).

(6-B) A heat exchanger provided in the air conditioner (1) may also serve as the temperature regulator.

### Other Embodiments

The embodiments and variations described above may be implemented as follows.

The ultraviolet light emitting device (10) may include a means configured to change the irradiation region (E1) of the ultraviolet rays emitted from the irradiator (20). Examples of the means include a moving part, a shield, and a control circuit. The moving part changes the position and orientation of the light source of the irradiator (20). The shielding member (60) physically shields part of the irradiation region of the irradiator (20). The control circuit controls the irradiator (20) to enlarge or shrink the irradiation region (E1). The recognizer notifies the subject (H) of where in the target space (S) the changeable irradiation region (E1) is located.

The ultraviolet light emitting device (10) may be provided on a front surface of a wall-mounted air conditioner.

The air treating device may be an air cleaner, a dehumidifier, a humidifier, a ventilator, or any other similar device.

The ultraviolet light emitting device (10) does not necessarily have to be provided for the air treating device. The ultraviolet light emitting device (10) may be independently provided on the ceiling or a wall of a building. The ultraviolet light emitting device (10) may be provided in a mobile object, such as a vehicle or a train.

While the embodiments and the variations thereof have been described above, it will be understood that various changes in form and details may be made without departing from the spirit and scope of the claims. The embodiments, the variations, and the other embodiments may be combined and replaced with each other without deteriorating intended functions of the present disclosure.

The ordinal numbers such as “first,” “second,” “third,” . . . , described above are used to distinguish the terms to which these expressions are given, and do not limit the number and order of the terms.

### INDUSTRIAL APPLICABILITY

As can be seen from the foregoing description, the present disclosure is useful for an ultraviolet light emitting device and an air treating device.

### DESCRIPTION OF REFERENCE CHARACTERS

10 Ultraviolet Light Emitting Device
20 Irradiator
30 Light Emitter (Recognizer)
40 Detector
60 Shielding Member
72 Lens (Light Guide Member)
73 Reflector (Light Guide Member)
75 Light Blocking Member (Light Guide Member)
77 Slit (Hole)

1. An ultraviolet light emitting device, comprising:
an irradiator configured to emit ultraviolet light into a target space (S) where a subject is present,
a recognizer configured to notify the subject of a state of the ultraviolet light emitted from the irradiator,
a controller configured to change an intensity of the ultraviolet rays emitted from the irradiator, wherein
the recognizer includes a light emitter configured to emit visible light, and
the light emitter changes an illuminance or a hue of the visible light in accordance with the intensity of the ultraviolet light emitted from the irradiator.

2. An ultraviolet light emitting device, comprising:
an irradiator configured to emit ultraviolet light into a target space (S) where a subject is present,
a recognizer configured to notify the subject of a state of the ultraviolet light emitted from the irradiator,
the recognizer includes a light emitter configured to emit visible light,
the light emitter changes an illuminance or a hue of the visible light in accordance with the irradiation cumulative amount of the ultraviolet light emitted from the irradiator.

3. The ultraviolet light emitting device of claim 1, wherein
the light emitter emits the visible light such that the visible light overlaps with the irradiation region of the ultraviolet light emitted from the irradiator.

4. The ultraviolet light emitting device of claim 3, wherein
the light emitter emits the visible light such that the irradiation region of the ultraviolet light emitted from the irradiator overlaps with the whole region of visible light.

5. The ultraviolet light emitting device of claim 1, wherein
the light emitter emits the visible light such that the visible light indicates a boundary of the irradiation region of the ultraviolet light emitted from the irradiator.

6. The ultraviolet light emitting device of claim 1, further comprising:
a shielding member configured to shield the light emitter from a sight of the subject outside the irradiation region of the ultraviolet light emitted from the irradiator.

7. The ultraviolet light emitting device of claim 1, further comprising:
a light guide member configured to guide the ultraviolet light emitted from the irradiator and the visible light emitted from the light emitter in a same direction.

8. The ultraviolet light emitting device of claim 7, wherein
a distance L1 between the irradiator and the light emitter is shorter than an average L2 of a shortest distance between the irradiator and the light guide member and a shortest distance between the light emitter and the light guide member.

9. The ultraviolet light emitting device of claim 7, wherein
the light guide member includes a lens configured to change a direction of the ultraviolet light and a direction of the visible light.

10. The ultraviolet light emitting device of claim 7, wherein
the light guide member includes a reflector configured to reflect the ultraviolet light and the visible light.

11. The ultraviolet light emitting device of claim 7, wherein
the light guide member includes a light blocking member having a hole through which the ultraviolet light and the visible light pass.

12. The ultraviolet light emitting device of claim 1, wherein
the light emitter is an LED.

13. The ultraviolet light emitting device of claim 1, further comprising:
a detector configured to detect the subject, wherein
if the detector detects the subject, the recognizer notifies the subject of the state of the ultraviolet light.

14. The ultraviolet light emitting device of claim 1, wherein
the ultraviolet light emitted from the irradiator has a wavelength of from 190 nm or more to 280 nm or less.

15. The ultraviolet light emitting device of claim 1, wherein
the ultraviolet light emitted from the irradiator has a wavelength of from 190 nm or more to 230 nm or less.

16. An air treating device, comprising:
the ultraviolet light emitting device of claim 1.
